(19) United States
(12) Patent Application Publication
(10) Pub. No.: US 2012/0165761 A1
(43) Pub. Date: Jun. 28, 2012

(54) DRUG ELUTION THERAPY APPARATUS AND METHOD FOR TREATING BLOOD VESSELS

(76) Inventor: Ray Hill, Santa Rosa, CA (US)

(21) Appl. No.: 12/979,285

(22) Filed: Dec. 27, 2010

(51) Int. Cl. A61M 31/00

(52) U.S. Cl. 604/285

(57) ABSTRACT

A drug therapy elution system and method for treating blood vessels. The system includes an enclosed receptacle containing the drug therapy for treating the damaged blood vessel. The system also includes one or more conduits that permit elution of the drug therapy from the enclosed receptacle into the damaged blood vessel wall. The system also includes one or more fixation mechanisms that can secure the enclosed receptacle to the blood vessel wall.

### BACKGROUND OF THE INVENTION

The present invention relates generally to medical device systems and methods and more specifically to medical device systems and methods for treating defects in the human circulatory system.

Our national healthcare expenditure is expected to increase by billions of dollars within the next few years particularly in light of the current increased life expectancy rate. Increased costs are expected for many medical conditions such as cardiovascular and blood vessel disorders, mental health conditions, mobility disability, etc.

Despite such increased healthcare expenditure, some medical conditions can be fatal. As an example, damaged blood vessels can cause ischemia or myocardial infraction, which is one of the leading causes of fatalities in the United States.

Aneurysms can also be fatal as well. A common aneurysm occurs when muscle fibers of the tunica media are weak. When blood flows through an affected area, the arterial wall may bulge and sometimes rupture. An aneurysm patient can receive a graft stent or metal tube to cover the weakened portion of the arterial wall so that blood flows through and does not reach the weakened wall area. In some cases, however, the stent graft can move downwards from its proper position exposing the arterial wall to blood-flow, which can become fatal.

An alternative approach to treatment is early detection. The objective of which is to detect a medical condition early so that drug therapy is administered before the medical condition can deteriorate.

It is within the above-described context that a need for the present invention has arisen and the present invention meets this need by addressing one or more of the foregoing disadvantages.

### BRIEF SUMMARY OF THE INVENTION

Various aspects of a drug therapy elution system and method can be found in exemplary embodiments of the present invention.

In a first embodiment, a system for delivering drug therapy to a damaged blood vessel is disclosed. The system includes an enclosed receptacle that can fit within the damaged blood vessel, the enclosed receptacle containing the drug therapy for treating the damaged blood vessel.

Here, note that the drug therapy contained therein can be a pharmacological agent for treating the damaged blood vessel. Alternatively, the drug therapy can also be a biologically active agent for effectuating blood vessel treatment. The enclosed receptacle of the present invention has an exterior wall or housing that runs between a proximal and a distal end having a substantially closed surface.

The system also includes a conduit extending from an interior of the enclosed receptacle to the closed surface at the distal end. The conduit allows elution of the drug therapy from the enclosed receptacle into the damaged blood vessel wall. Depending upon the desired elution rate profile, additional conduits can be included within the system.

The system of the present invention also includes one or more fixation mechanisms attached to the distal end of the enclosed receptacle. The fixation mechanism might include barbs, hooks or the like that can secure the enclosed receptacle to the blood vessel wall. In this manner, the blood vessel wall can remain on the blood vessel wall for an extended time duration as necessary for completion of the drug therapy treatment.

Unlike conventional stents, the enclosed receptacle held in place by the fixation mechanisms with little risk that the enclosed receptacle will move downwards resulting in a fatality. Multiple elution systems are moreover used to provide a fail proof system such that one system can continue operation in the event of failure by another system.

The system of the present invention also facilitates treatment for early detection cases such as when a detected aneurysm is too small to be operable. The present invention can be employed to administer drug therapy before the medical condition can deteriorate.

A further understanding of the nature and advantages of the present invention herein may be realized by reference to the remaining portions of the specification and the attached drawings. Further features and advantages of the present invention, as well as the structure and operation of various embodiments of the present invention, are described in detail below with respect to the accompanying drawings. In the drawings, the same reference numbers indicate identical or functionally similar elements.

### BRIEF DESCRIPTION OF THE DRAWINGS

FIG. 1A illustrates an elution system according to an exemplary embodiment of the present invention.

FIG. 1B is a front plan view of the elution system of FIG. 1A according to an exemplary embodiment of the present invention.

FIG. 2A illustrates an elution system according to an exemplary embodiment of the present invention.

FIG. 2B illustrates exemplary embodiments of pin heads for use with fixation mechanisms according to an exemplary embodiment of the present invention.

FIG. 3A is a top plan view of a damaged blood vessel to which multiple elution systems are attached according to exemplary embodiments of the present invention.

FIG. 3B is a front cross-sectional view of the damaged blood vessel and the multiple elution systems of FIG. 3A.

### DETAILED DESCRIPTION OF THE INVENTION

Reference will now be made in detail to the embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention.

However, it will be obvious to one of ordinary skill in the art that the present invention may be practiced without these specific details. In other instances, well-known methods, procedures, components, and circuits have not been described in detail as to not unnecessarily obscure aspects of the present invention.

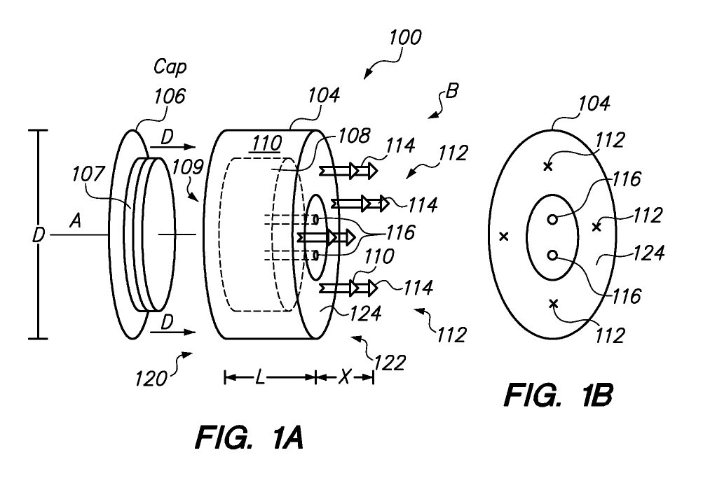

FIG. 1A illustrates elution system 100 according to an exemplary embodiment of the present invention.

In FIG. 1A, a user can utilize elution system 100 for treating damaged blood vessels. An example of a damaged blood vessel is an aneurysm, i.e., a dilation or bulging of a blood vessel caused by weakening of the blood vessel wall. Extended application of a drug therapy using elution system 100 according to the present invention can treat such damaged blood vessels.

Among other components, elution system 100 comprises receptacle 104 and cap 106 adapted to mate with and close receptacle 104. Although receptacle 104 is illustrated as being cylindrical shape, receptacle 104 can be any shape consistent with the spirit and scope of the present invention.

In one embodiment, receptacle 104 includes chamber 108 configured to receive and store drug therapy. As used herein, the term drug therapy may include any biologically active or pharmacological agent, either coated or non-coated or a mixture or suspension, in a solid, semi-solid, liquid, semi-liquid, gaseous or in any other state intended for treatment of a defective blood vessel. As an example, the drug therapy might be a heparin silicone composition having a suitable cross linker that affects the release rate profile.

The drug therapy provided herein is merely exemplary and is not intended to limit the type of substance contained within receptacle 104. The substance contained therein can be any biologically active or pharmacological agent for introduction into a body lumen for treatment purposes.

Receptacle 104 is defined by exterior wall 110 running from proximal end 120 to distal end 122. In particular, exterior wall 110 extends around a horizontal axis A running through a center point of receptacle 104. The thickness of exterior wall 110, that is, the length from chamber 108 walls to exterior wall 110 is preferably 0.04 inches (1 mm).

As shown, proximal end 120 includes aperture 109 through which the drug therapy is received. Once drug therapy is received, cap 106 is utilized to seal chamber 108. Specifically, cap 106 includes male grooves 107 adapted to mate with corresponding female grooves (not shown) within receptacle 106 to close and seal aperture 109.

As shown, distal end 120 is itself defined by front surface 124. Front surface 124 is a wall of appropriate thickness depending upon the conduits provided therein as further described below.

The dimensions and size of receptacle 104 depend upon the blood vessel being treated. Such blood vessels can range from 8 μm-25 mm (000314 inches-1 inch) for an aorta. In one embodiment, the length L from a proximal end 120 to distal end 122 is 0.25 inches (0.635 cm). An exemplary diameter D is 0.25 inches (0.635 cm). The capacity of receptacle 104 depends upon the amount of drug therapy required for treatment.

Receptacle 104 can be injection-molded and comprise of any polymeric or biocompatible material. Examples can be PTFE (polytetraflouroethylene), PVC (polyvinyl chloride) PEEK (polyetheretherketone) or other biodegradable polymers. Note that receptacle 104 is dimensioned to fit within a blood vessel without holding the blood vessel open unlike a traditional stent. As will be further discussed, receptacle 104 is attachable to a blood vessel without obstructing blood flow within the blood vessel.

In FIG. 1A, elution system 100 further comprises one or more fixation mechanisms 112 that are attached to distal end 122. In an embodiment, fixation mechanisms 112 might be unitarily formed with receptacle 104. Alternatively, fixation mechanisms 112 can be attached by screwing, welding, joining or other suitable fastening techniques. If fixation mechanisms 112 and receptacle 104 are unitarily formed, then fixation mechanisms 112 can be made of the same receptacle 104 material as mentioned above. Fixation mechanisms 112 can also be made of acrylic, silicon resins and other materials consistent with the spirit and scope of the present invention.

Each of fixation mechanisms 112 itself might be pins or other fastening having a plurality of barbs 114 that are intended to go through and clasp a blood vessel wall to retain receptacle 104 onto said blood vessel wall for an extended time duration. In one embodiment, plurality of barbs 114 is not unlike the barbs found on bee stingers.

The length X of each of fixation mechanisms 112 is of course dependent upon the thickness of the blood vessel wall. In some instances, as in the case of the aorta, X might be about 25 millimeters in order for each fixation mechanism 112 to securely penetrate the aorta and remain for an extended duration of time.

The number of fixation mechanisms 112 utilized can also vary, but it is preferred that at least two of such mechanisms that are oppositely disposed be utilized so that receptacle 104 is securely fastened to a blood vessel wall. Although shown with plurality of barbs 114, a single barb located at the head of each fixation mechanism can also be utilized.

Fixation mechanisms 112 can be made of suitable material consistent with the spirit and scope of the present invention. Such material might include but is not limited to high-grade surgical stainless steel 316L, woven polyester and cobalts-chromium. In such a case, receptacle 104 will also be made of material corresponding to and compatible with fixation mechanism 112. The diameter of each fixation mechanism 112 is preferably 0.0394 inches (1 mm).

As noted, it is preferable that at least two fixation mechanisms be utilized. An advantage of the present invention is that such fixation mechanisms unlike conventional stents are secured to the blood vessel walls at multiple contact points. Unlike such conventional stents which are typically not secured onto the vessel wall, the present invention uses fixation mechanisms to secure receptacle 104 onto the vessel walls such that receptacle 104 can remain on a blood vessel for extended time periods until drug therapy is completely dispensed.

In FIG. 1A, elution system 100 further comprises a plurality of conduits 116. Plurality of conduits 116 extends from front surface 124 of distal end 122 into chamber 108. Specifically, conduits 116 extend from the interior of chamber 108 through the surface of exterior wall to front surface 124. Conduits 116 permit elution of drug therapy that is located within chamber 108.

Preferably, the diameter of each of conduits 116 is about 5 μm but is nevertheless dependent upon the desired elution rate. The higher the elution rate desired, the larger the diameter of conduit 116. Moreover, the elution rates can also be impacted by the number of conduits provided by elution system 100. Other factors such as the amount of crosslinking material in the drug therapy and the drug therapy particle size can also impact the desired release rate profile. Here, note that although only two conduits 116 are shown, more or less conduits can be utilized, again, depending upon the elution rate desired.

Although not shown, each conduit 116 might also include a filter within either within or outside of each conduit. The filter can also impact the elution rate profile. In one embodiment, the filter might be a mesh consisting of a biocompatible material.

FIG. 1B is a front plan view of elution system 100 according to an exemplary embodiment of the present invention.

In FIG. 1B, specifically, a view of front surface 124 from a direction B is shown. Front surface 124 includes conduits 116 as well as the plurality of fixation mechanisms 112. Each of fixation mechanisms 112 are appropriately dispersed around front surface 124.

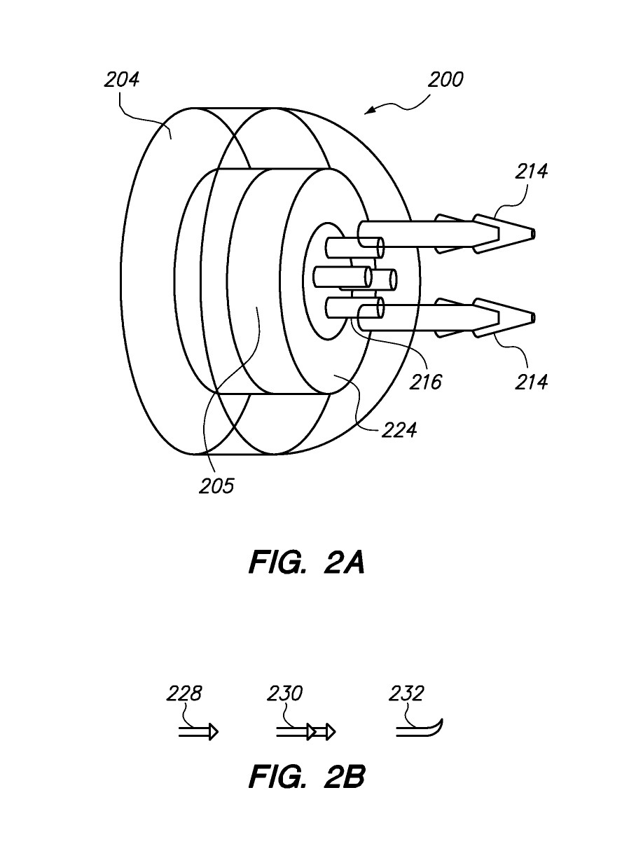

FIG. 2A illustrates elution system 200 according to an exemplary embodiment of the present invention.

In FIG. 2A, elution system 200 includes housing 204 and chamber 205. In FIG. 2A, unlike embodiment of FIG. 1A, housing 204 is separately formed and serves to protect chamber 205. A plurality of barbs 214 is also shown. Here, barbs 214 are conically shaped and are located closer to each other such that a more secure penetration is achieved into the wall of a blood vessel. As shown, FIG. 2A also includes a plurality of conduits 216 running from chamber 205 through housing 204 to from surface 224.

FIG. 2B illustrates exemplary embodiments of pin heads for use with fixation mechanisms according to an exemplary embodiment of the present invention.

In FIG. 2B, single pin head 228 may be utilized as the pin head for each of fixation mechanisms 214. Alternatively, a plurality of pin heads 230 can be used. Further yet, a curved hook 232 may be employed as the pin head for each of fixation mechanisms 214. Although not shown, one skilled in the art will realize that other comparable pin heads consistent with the spirit and scope of the present invention can be employed.

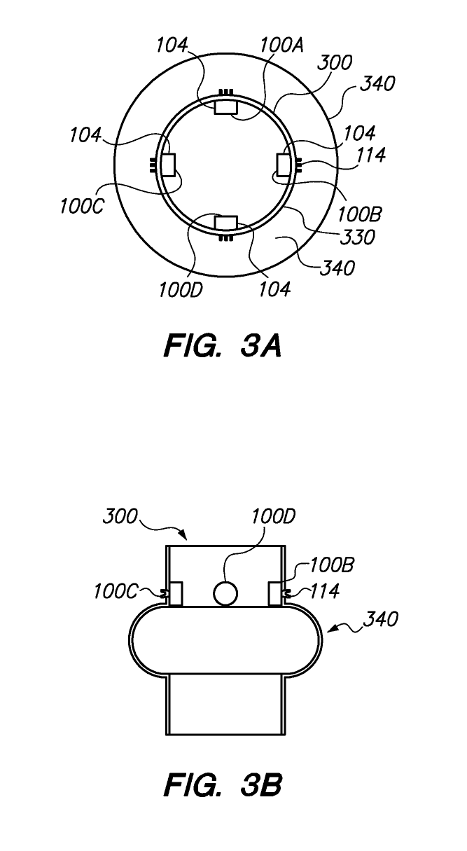

FIG. 3A is a top plan view of damaged blood vessel 300 to which multiple elution systems are attached according to exemplary embodiments of the present invention.

In FIG. 3A, multiple elution systems namely elution system 100A, 100B, 100C and 100D are attached to damaged blood vessel 300. Here, damaged blood vessel 300 is characterized by an aneurysm 340. Here, the elution systems are attached above aneurysm 340 so that drug therapy can be eluted within the aneurysm walls. As shown, each of fixation mechanisms 112 (FIG. 1) extends from receptacle 104 trough vessel wall 330 to firmly secure receptacle 104 to damaged blood vessel 300.

FIG. 3B is a front cross-sectional view of damaged blood vessel 300 and elution systems 100A, B, C and D of FIG. 3A.

In FIG. 3B, the elution systems are shown attached over aneurysm 340. Use and operation of elution system of the present invention will now be described with reference to FIGS. 1A, 1B, 2A, 2B, 3A and 3B.

In use, a user begins by providing the desired drug therapy within chamber 108 of FIG. 1A and thereafter using cap 106 to seal and secure the drug therapy within receptacle 104. As previously noted, this drug therapy may include any biologically active or pharmacological agent.

After chamber 108 is filled with the desired drug therapy and secured therein, receptacle 104 is then ready for placement into damaged blood vessel 300 above aneurysm 340 (FIG. 3A). In FIGS. 3A and 3B, elution systems 100A, 100B, 100C, 100D are placed by using a catheter (not shown). Specifically, the elution systems are stacked into an elongated catheter shaft have a tab at the distal end, said tab releasing only a single elution system when a force is applied at a proximal end of the catheter. A drive means on the catheter is also used to implant each released elution system into blood vessel wall 330. In the catheter, the stacked elution systems align so that they are linear to the forces of engagement that secure them to blood vessel wall 330.

Note that although not shown, receptacle 104 can also be attached to the exterior side of blood vessel wall 330 via surgery. In such a case, a physician can perform surgery to directly insert elution system 100 into damaged blood vessel 300 without employing a catheter.

The result is that each of a plurality of elution systems 100A, 100B, 100C and 100D is attached to blood vessel wall 330 of FIG. 3B. As can be seen, the pin heads of fixation mechanism 112 protrude through blood vessel wall 330 to secure each elution system to blood vessel wall 330.

Once each elution system 100A, 100B, 100C and 100D is secured onto blood vessel wall 330, elution of drug therapy via each of conduits 116 into blood vessel wall 330 can begin. The elution occurs by extraction or release by direct contact of bodily fluids (and/or blood flow) through each of conduits 116 with the drug therapy in chamber 108. The eluted drug therapy is localized around the blood vessel wall 330. Once eluted, the drug therapy can provide nutrients (such as to the blood vessel wall elastin) that is located within blood vessel wall 330 such that strength and elasticity can be regained.

In the case of an aneurysm, the elasticity of the surrounding walls is corrected, and the weakened walls are strengthened in order to treat the aneurysm. An advantage of the present invention is that the fixation mechanisms 116 can securely hold the attached receptacle 104 and the drug therapy contained therein onto the blood vessel wall.

Unlike conventional stents, receptacle 104 is held in place by a plurality of fixation mechanisms with little risk that the receptacle will move downwards resulting in a fatality. Multiple elution systems are moreover used to provide a fail proof system such that one system can remain operation even if another elution system fails. In this manner, the present invention provides one or more advantages not seen or witnessed by conventional prior art systems.

While the above is a complete description of exemplary specific embodiments of the invention, additional embodiments are also possible. Thus, the above description should not be taken as limiting the scope of the invention, which is defined by the appended claims along with their full scope of equivalents.

I claim:

1. An apparatus for delivering a drug therapy to a damaged blood vessel, said apparatus comprising:
an enclosed receptacle dimensioned to fit within a blood vessel without propping said blood vessel open, wherein said enclosed receptacle is defined by an exterior wall extending between a proximal end and a distal end of said enclosed receptacle, said enclosed receptacle storing the drug therapy applicable to treat said blood vessel;
one or more fixation mechanisms attached to the distal end of said enclosed receptacle, said fixation mechanism securing the enclosed receptacle to a wall of said blood vessel such that the enclosed receptacle remains locatable on the wall for an extended time duration; and
a conduit extending from an interior of the enclosed receptacle through the exterior wall at said distal end, wherein upon said enclosed receptacle being attached to said blood vessel wall by said fixation mechanism, said conduit permitting elution of the drug therapy from the enclosed receptacle to the blood vessel.

2. The apparatus of claim 1 wherein said fixation mechanism is a barbed device.

3. The apparatus of claim 1 wherein said fixation mechanism is a hooked device.

4. The apparatus of claim 1 wherein said proximal end includes an opening.

5. An apparatus comprising:
a housing dimensioned to be attachable to a blood vessel, wherein said housing is defined by an exterior wall extending between a proximal end and a distal end of said housing, said housing storing the drug therapy applicable to treat said blood vessel;
one or more fixation mechanisms attached to the distal end of said housing, said fixation mechanism securing the housing to a wall of said blood vessel such that the housing remains locatable on the wall for an extended time duration; and
a conduit extending from an interior of the housing through the exterior wall at said distal end, wherein upon said housing being attached to said blood vessel wall by said fixation mechanism, said conduit permitting elution of the drug therapy from the housing to the blood vessel.

6. The apparatus of claim 5 wherein said fixation mechanism is a barbed device.

7. The apparatus of claim 5 wherein said fixation mechanism is a hooked device.

8. The apparatus of claim 5 further comprising a cap for sealing an opening at the proximal end, said opening for receiving the drug therapy.

9. The apparatus of claim 5 wherein said conduit is about 2 microns in diameter.

10. The apparatus of claim 5 wherein said elution of the drug therapy is localized in the wall of the blood vessel.

11. The apparatus of claim 5 wherein the drug therapy is a coating within said receptacle.

12. The apparatus of claim 5 wherein the drug therapy is semi-solid.

13. The apparatus of claim 5 wherein the drug therapy is solid.

14. A method for delivering a drug therapy to a damaged blood vessel, said method comprising:
providing an enclosed receptacle dimensioned to fit within a blood vessel without propping said blood vessel open, wherein said enclosed receptacle is defined by an exterior wall extending between a proximal end and a distal end of said enclosed receptacle, said enclosed receptacle storing the drug therapy applicable to treat said blood vessel;
using one or more fixation mechanisms to secure the enclosed receptacle to a wall of said blood vessel such that the enclosed receptacle remains locatable on the wall for an extended time duration; and
using a conduit extending from an interior of the enclosed receptacle through the exterior wall at said distal end to elute the drug therapy from the enclosed receptacle to the blood vessel when enclosed receptacle is attached to said blood vessel wall by said fixation mechanism.

15. The method of claim 14 wherein said fixation mechanism is a barbed device.

16. The apparatus of claim 14 wherein said fixation mechanism is a hooked device.
